(19) United States
(12) Patent Application Publication
(10) Pub. No.: US 2007/0276431 A1
(43) Pub. Date: Nov. 29, 2007

(54) Surgical box hinge and method of making same

(76) Inventors: Jennifer T. Swartz, Libertyville, IL (US); James P. Moorman, Lake Forest, IL (US)

Correspondence Address: NIXON PEABODY, LLP, 161 N. CLARK ST., 48TH FLOOR, CHICAGO, IL, 60601-3213, US

(21) Appl. No.: 11/805,785

(22) Filed: May 24, 2007

Related U.S. Application Data
(60) Provisional application No. 60/809,016, filed on May 26, 2006.

(51) Int. Cl. A61B 17/00

(52) U.S. Cl. 606/208

(57) ABSTRACT

A surgical instrument including a male portion and a female portion. The male portion is opposably coupled to the female portion. Both the male portion and the female portion include a working end, a handle end, and a box hinge portion between the handle end and the working end. Both the male portion and the female portion include a first ramp between the box hinge portion and the working end. Also, both the male portion and the female portion include a second ramp between the box hinge portion and the handle end. An opening is formed by the male and female portions near the working end when the surgical instrument is in a closed position.

### CROSS-REFERENCE TO RELATED APPLICATION

This application claims the benefit of U.S. Provisional Application No. 60/809,016 filed May 26, 2006, which is hereby incorporated by reference in its entirety. This application also claims the benefit of application Ser. No. 29/260,281 filed May 23, 2006.

### FIELD OF THE INVENTION

The present invention relates generally to medical instruments and more particularly to medical instruments such as suture holders which incorporate a box hinge.

### BACKGROUND OF THE INVENTION

Medical instruments that incorporate a box hinge are well-known. These instruments include suture holders used for holding sutures during surgery. These instruments include a male portion and a female portion. The female portion includes a slot that is designed to receive the male portion. The slot is defined by two parallel pieces. The male and female portions are generally joined together at a pivot point, which enables the male and female portions to pivot relative to each other. In some prior art instruments, the male portion includes an edge machined to conform to the parallel pieces of the female portion. However, these machined edges can snag sutures above and below the pivot point of the hinge. Snagging can interfere as a surgeon or other medical technician is tying knots in the suture. Also, machining is an expensive portion of the manufacturing process.

Therefore, there is a need for a box hinge for use in surgical instruments that do not snag sutures.

### SUMMARY OF THE INVENTION

According to one embodiment of the present invention, a surgical instrument is provided that includes a first portion. The first portion includes a first working end, a first handle end, and a first box hinge portion between the first working end and the first handle end. The surgical instrument also includes a second portion opposingly coupled to the first portion. The second portion includes a second working end, a second handle end, and a second box hinge portion located between the second handle end and the second working end. The first box hinge portion includes an elongated slot for receiving the second box hinge portion. The first portion has a first ramp extending downwardly from the first box hinge portion toward the first working end and a second ramp extending downwardly from the first box hinge portion toward the first handle end. The second portion has a third ramp extending downwardly from the second working end toward the second box hinge portion and a fourth ramp extending downwardly from the second handle end toward the second box hinge portion. An opening is formed by the first and second portions near the first and second working ends when the surgical instrument is in a closed position, the opening being partially formed by the third ramp.

According to another embodiment of the present invention, a method of making a surgical instrument is provided. The method includes providing a first portion having a first working end, a first handle end, and a first box hinge portion located between the first working end and the first handle end. The first box hinge portion includes an elongate slot. The method also includes providing a second portion, the second portion including a second working end, a second handle end, and a second box hinge portion located between the second handle end and second working end. The second box hinge portion is inserted through the elongate slot of the first box hinge portion and pivotally attaching the second box hinge portion to the first box hinge portion. A first ramp is provided on the first portion, such that the first ramp extends downwardly from the first box hinge portion toward the first working end. A second ramp is provided and extends downwardly from the first box hinge portion toward the first handle end. A third ramp is provided on the second portion such that the third ramp extending downwardly from the second working end toward the second box hinge portion. A fourth ramp is provided and extends downwardly from the second handle end toward the second box hinge portion. An opening formed by the first and second portions near the first and second working ends when the surgical instrument is in a closed position is provided and the opening being partially formed by the third ramp.

According to yet another embodiment of the present invention, a surgical instrument including a male portion and a female portion is provided. The male portion is opposingly coupled to the female portion. Both the male portion and the female portion include respective working ends, respective handle ends, and respective box hinge portions located between the working end and the handle end. Both the male portion and the female portion include a first ramp between the respective box hinge portion and the respective working end. Both the male portion and the female portion also include a second ramp between the respective box hinge portion and the respective handle end. An opening is formed by the male and female portions near the working end when the surgical instrument is in a closed position.

The above summary of the present invention is not intended to represent each embodiment or every aspect of the present invention. The detailed description and figures will describe many of the embodiments and aspects of the present invention.

### BRIEF DESCRIPTION OF THE DRAWINGS

The foregoing and other advantages of the invention will become apparent upon reading the following detailed description and upon reference to the drawings.

FIG. 1 shows a top view of a suture holder having a box hinge according to one embodiment of the present invention;

FIG. 2 shows a perspective view of the suture holder of FIG. 1 in an open position;

FIG. 2A shows a blown-up view; and

FIG. 3 illustrates a side view of the suture holder of FIG. 1.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

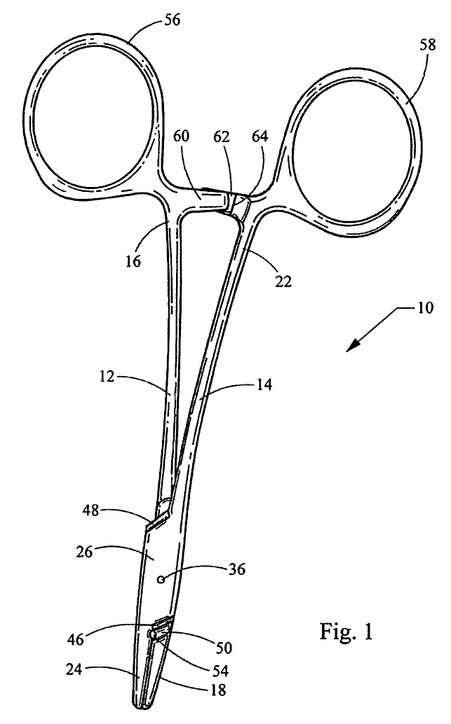

Turning now to FIGS. 1-3, a suture holder 10 having a male portion 12 and a female portion 14 is illustrated. The male portion 12 includes a handle end 16 and a working end 18. In between the handle end 16 and the working end 18, the male portion 12 includes a box hinge portion 20. The female portion 14 includes a handle end 22 and a working end 24. In between the handle end 22 and the working end 24 of the female portion 14 is a box hinge portion 26.

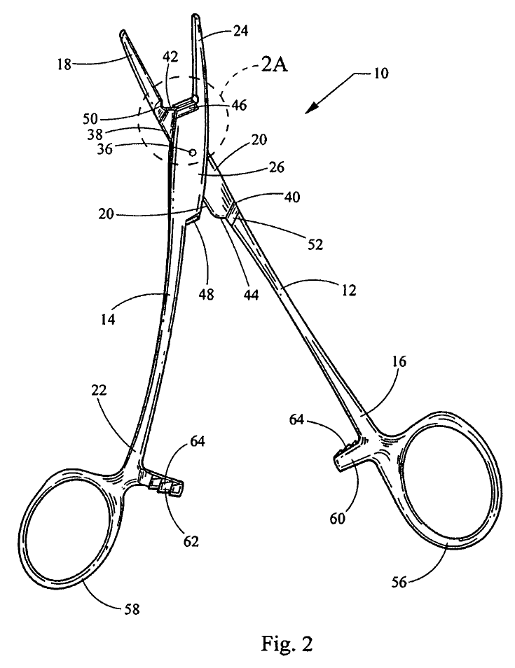

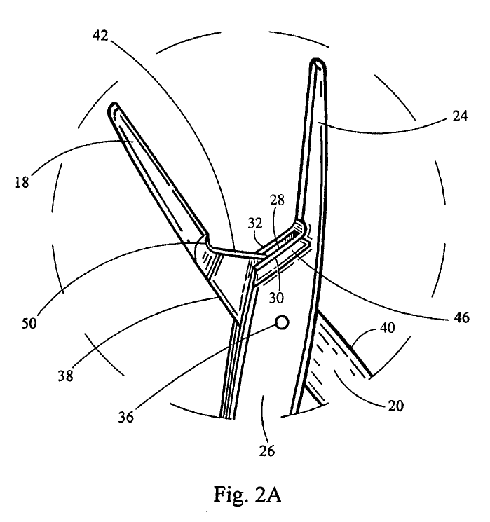

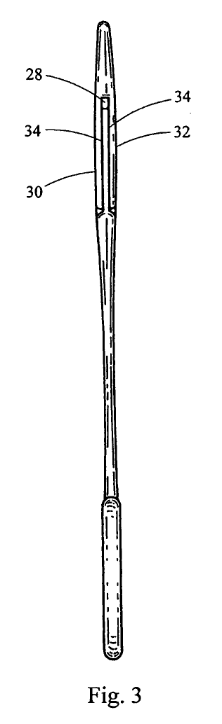

The box hinge portion 26 of the female portion 14 includes a slot 28 (FIGS. 2A and 3) that is defined by two generally parallel sides 30, 32. The generally parallel sides 30, 32 include edges 34. The generally parallel sides 30, 32 are designed to receive the box hinge portion 20 of the male portion 12. The box hinge portion 20 of the male portion 12 and the box hinge portion 26 of the female portion 14 are opposingly coupled and/or pivotally attached. In the illustrated embodiment, the male portion 12 and female portion 14 are connected via a hinge pin 36 (FIGS. 1, 2 and 2A). In other embodiments, other suitable types of connection devices may be used to connect the box hinge portion 20 of the male portion 12 and the box hinge portion 26 of the female portion 14.

As shown in FIGS. 2, 2A and 3, the box hinge portion 20 of the male portion 12 includes a single piece defined by a pair of generally parallel edges 38, 40. One generally parallel edge 40 blends to a curved edge 42 near the working end 18 of the male portion 12. The other generally parallel edge 38 blends to a curved edge 44 near the handle end 16 of the male portion 12. As shown in FIG. 2, the box hinge portion 20 of the male portion 12 is wider than both the working end 18 and the handle end 16 of the male portion 12. In FIG. 3, it can be seen how the curved edge 42 (of FIGS. 2-2A) near the working end 18 of the male portion 12 creates a cross-section that does not entirely conform to the generally parallel sides 30, 32 of the female portion 14. In other words, because the parallel edge 40 curves at the curved edge 42, the edge of the male portion 12 is not completely flush with the edge of the female portion 14.

The edge 34 of the female portion 14 includes a ramp 46 that slopes downwardly from the box hinge portion 26 toward the working end 24. The other edge 34 of the female portion 14 includes a ramp 48 that slopes downwardly from the box hinge portion 26 toward the handle end 22. The ramps 46, 48 are useful in that sutures that come into contact with the ramps slide over the ramps, preventing snagging.

The box hinge portion 20 of the male portion 12 also includes a ramp 50 that slopes downwardly from the working end 18 toward the box hinge portion 20. The box hinge portion 20 of the male portion 12 also includes a ramp 52 that slopes downwardly from the handle end 22 toward the box hinge portion 20. As illustrated, the ramps 46, 48 of the female portion 14 slope downward toward the ramps 50, 52 of the male portion 12. In the illustrated embodiment, the ramps 50, 52 of the male portion 12 are longer and less steep than the ramps 46, 48 of the female portion 14. However, in other embodiments, the ramps 50, 52 of the male portion 12 may be equally as steep or steeper than the ramps 46, 48 of the female portion 14. Also, in other embodiments, the ramps 50, 52 of the male portion 12 may be shorter or equal in length to the ramps 46, 48 of the female portion 14.

As shown in FIG. 1, when the suture holder 10 is in a closed position, the box hinge portion 26 forms a hole 54 between the male portion 12 and the female portion 14. The hole 54 is formed along the ramp 50 of the male portion 12 such that when a suture slides over the ramp 50, it can slip through the hole 54.

The working ends 18, 24 of the male and female portions 12, 14 each have a circular handle 56, 58, respectively. The circular handles 56, 58 are used for grasping and manipulating the suture holder 10 between an open and a closed position. The working ends 18, 24 also include complementary locking mechanisms 60, 62. The locking mechanisms 60, 62 interact to lock the suture holder 10 in a closed position. In the depicted embodiment, the locking mechanisms 60, 62 include multiple opposing stepped protrusions 64 so the suture holder 10 could be locked in multiple positions. Other embodiments may include other types of suitable locking mechanisms.

Although the present invention has been described as a suture holder, it should be understood that the present invention may be used in connection with a wide variety of surgical instruments including, but not limited to: clamp-type forceps, blood vessel clamps, scissors, or other manual surgical instruments that employ box hinges.

During use of a suture holder, a surgeon or other medical professional uses the suture holder along with a suture and needle to suture a wound or other opening. The suture is operatively mounted in the working end of the suture holder and the wound or other opening is sutured according to conventional medical practice.

In a one embodiment, the suture holder 10 is manufactured of stainless steel. However, other materials such as nickel, chrome, steel, brass, and/or combinations thereof may also be used. The handles of the suture holder are generally round in shape; however, other shapes conducive to grasping, such as oval, square, etc. may also be used.

The suture holder 10 is manufactured by melting down the raw material and pouring it into casting blocks. The casting blocks are then heated and run through a press, creating the thickness of the material. The material is then cut into strips and the form of the suture holders are cut out, creating male and female portions. The strips of material are also stamped to the correct size and shape. Any holes, sharp angles, or flat surfaces are then machined, or smoothed. The male and female portions are then assembled and ground down to the correct specifications.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention, which is set forth in the following claims.

What is claimed is:

1. A surgical instrument comprising:
a first portion having a first working end, a first handle end, and a first box hinge portion between the first working end and the first handle end;
a second portion opposingly coupled to the first portion, the second portion having a second working end, a second handle end, and a second box hinge portion located between the second handle end and the second working end;
the first box hinge portion including an elongated slot for receiving the second box hinge portion;
the first portion having a first ramp extending downwardly from the first box hinge portion toward the first working end and a second ramp extending downwardly from the first box hinge portion toward the first handle end;
the second portion having a third ramp extending downwardly from the second working end toward the second box hinge portion and a fourth ramp extending downwardly from the second handle end toward the second box hinge portion; and
an opening formed by the first and second portions near the first and second working ends when the surgical instrument is in a closed position, the opening being partially formed by the third ramp.

2. The surgical instrument of claim 1, wherein both the first and second handle ends include handles for grasping the surgical instrument.

3. The surgical instrument of claim 1, wherein both the first and second handle ends include a locking mechanism for locking the surgical instrument in a particular position.

4. The surgical instrument of claim 3, wherein the locking mechanism includes a first lock on the first handle end and a second lock on the second handle end, the first lock and the second lock opposing one another.

5. The surgical instrument of claim 4, wherein the opposing first and second locks include a plurality of opposing, stepped protrusions such that the surgical instrument may be locked in a plurality of positions.

6. The surgical instrument of claim 1, wherein the second box hinge portion is wider than the second handle end and the second working end.

7. The surgical instrument of claim 1, wherein the second box hinge portion includes a first edge and a second edge generally parallel to the first edge.

8. The surgical instrument of claim 7, wherein the first edge blends to a first curved portion toward the second working end.

9. The surgical instrument of claim 7, wherein the second edge blends to a second curved portion toward the second handle end.

10. A method of making a surgical instrument comprising:
providing a first portion having a first working end, a first handle end, and a first box hinge portion located between the first working end and the first handle end, the first box hinge portion having an elongate slot;
providing a second portion, the second portion including a second working end, a second handle end, and a second box hinge portion located between the second handle end and second working end;
inserting the second box hinge portion through the elongate slot of the first box hinge portion and pivotally attaching the second box hinge portion to the first box hinge portion;
providing a first ramp on the first portion, the first ramp extending downwardly from the first box hinge portion toward the first working end and a second ramp extending downwardly from the first box hinge portion toward the first handle end;
providing a third ramp on the second portion, the third ramp extending downwardly from the second working end toward the second box hinge portion and a fourth ramp extending downwardly from the second handle end toward the second box hinge portion; and
providing an opening formed by the first and second portions near the first and second working ends when the surgical instrument is in a closed position, the opening being partially formed by the third ramp.

11. The method of claim 10, wherein the pivotally attaching comprises attaching the first box hinge portion to the second box hinge portion with a hinge pin.

12. The method of claim 10, further comprising providing the first and second handle ends with handles for grasping the surgical instrument.

13. The method of claim 10, further comprising providing the first and second handle ends with a locking mechanism for locking the surgical instrument in at least one position.

14. The method of claim 13, wherein the locking mechanism is adapted to lock the first and second handle ends in a plurality of positions.

15. A surgical instrument comprising:
a male portion and a female portion, the male portion being opposingly coupled to the female portion;
both the male portion and the female portion including a respective working end, a respective handle end, and a respective box hinge portion located between the working end and the handle end;
both the male portion and the female portion including a first ramp between the respective box hinge portion and the respective working end;
both the male portion and the female portion including a second ramp between the respective box hinge portion and the respective handle end; and
an opening formed by the male and female portions near the respective working ends when the surgical instrument is in a closed position.

16. The surgical instrument of claim 15, wherein the surgical instrument is selected from suture holders, clamp-type forceps, blood vessel clamps, and scissors.

17. The surgical instrument of claim 15, wherein the first female ramp extends downwardly from the female box hinge portion toward the female working end.

18. The surgical instrument of claim 15, wherein the second female ramp extends downwardly from the female box hinge portion toward the female handle end.

19. The surgical instrument of claim 15, wherein the first male ramp extends downwardly from the male working end toward the male box hinge portion.

20. The surgical instrument of claim 15, wherein the first male ramp extends downwardly from the male handle end toward the male box hinge portion.

21. The surgical instrument of claim 15, wherein the female box hinge portion includes an elongated slot for receiving the male box hinge portion.
